(19) United States
(12) Patent Application Publication
(10) Pub. No.: US 2021/0001109 A1
(43) Pub. Date: Jan. 7, 2021

(54) PHOTOTHERAPY SYSTEM TO PREVENT OR TREAT DRIVELINE INFECTIONS

(71) Applicant: TC1 LLC, St. Paul, MN (US)

(72) Inventors: Jasjit S. Johal, St. Paul, MN (US); Dan Phong Ha, St. Paul, MN (US); Julio C. Beltran, St. Paul, MN (US)

(21) Appl. No.: 16/891,355

(22) Filed: Jun. 3, 2020

Related U.S. Application Data
(60) Provisional application No. 62/870,337, filed on Jul. 3, 2019.

(51) Int. Cl. A61M 39/16; A61M 1/12; A61M 1/10; A61N 5/06

(52) U.S. Cl. CPC A61M 39/16; A61M 1/122; A61M 1/1008; A61M 2039/167; A61N 2005/0652; A61N 2005/0663; A61N 5/0624

(57) ABSTRACT

Disclosed herein is a blue light phototherapy system and methods of using the system to prevent and treat driveline bacterial infections, and particularly ventricular assist device driveline bacterial infections (Right Ventricular Assist Devices (RVAD) and Left Ventricular Assist Devices (LVAD)). The blue light phototherapy systems provide for both repeated and/or periodic antimicrobial treatment of soft tissue surrounding a driveline exit site on an individual. In many embodiments, the system includes one or more arrays of light emitting diodes (LED) of predominantly blue wavelengths in combination with a single or multi-piece biocompatible structure that captures the arrays and provides for a fixed distance from the arrays to the exit site tissue surface.

### CROSS REFERENCE TO RELATED APPLICATIONS

This application claims priority to provisional application Ser. No. 62/870,337, filed Jul. 3, 2019, which is incorporated by reference herein in its entirety.

### FIELD OF THE DISCLOSURE

The present disclosure relates generally to systems and methods for preventing and/or treating driveline infections at a driveline exit point on an individual. In particular, the present disclosure relates to a blue light phototherapy system and related methods for preventing and/or treating driveline infections at a ventricular assist device driveline exit point on an individual.

### BACKGROUND

Heart disease is a major health problem that claims many lives per year. After a heart attack or other major cardiac event, a small number of patients can be treated with medicines or other non-invasive treatment. A significant number of other patients can recover from a heart attack or other cardiac event if provided with mechanical circulatory support in timely manner.

In one conventional approach for treating patients, a blood pump is inserted into a heart chamber, such as into the left ventricle of the heart and the aortic arch, to assist the pumping function of the heart. Other known conventional applications involve providing for pumping venous blood from the right ventricle to the pulmonary artery for support of the right side of the heart. The object of the pump is to reduce the load on the heart muscle for a period of time allowing the affected heart muscle to recover and heal. Blood pumps may also be used in some cases for percutaneous coronary intervention.

A ventricular assist device (VAD), also referred to as a mechanical circulatory support device, is one type of mechanical blood pump that helps pump blood from the lower chambers of the heart (ventricles) to the body. VADs may be placed in the left, right, or both ventricles of the heart, although they are most commonly used in the left ventricle. The VAD is connected to a control unit through a driveline that is used to drive the pump. The driveline enters/exits the patient's body a driveline exit point or port.

In some cases, the driveline exit point may be prone to infection due to its percutaneous nature. Although these driveline-type infections are generally temporary and treatable with conventional antibiotics, other preventive/treatment options may be desirable in some cases.

### BRIEF SUMMARY OF THE DISCLOSURE

In one embodiment, the present disclosure is directed to a blue light phototherapy system for treating or preventing an infection at a driveline exit site on an individual. The system comprises: (i) a standoff base structure sized and configured for insertion and removal around a driveline cable at the driveline exit site of the individual; and (ii) a cap structure sized and configured for attachment to the standoff base structure. The cap structure includes a light emission source capable of providing light in the range of from 405 nanometers to 475 nanometers.

In another embodiment, the present disclosure is directed to a blue light phototherapy system for treating or preventing an infection at a driveline exit site on an individual. The system comprises: (i) a biocompatible polymeric structure sized and configured for insertion and removal around a driveline cable at the driveline exit site of the individual. The biocompatible polymeric structure includes a light emission source capable of providing light in the range of from 405 nanometers to 475 nanometers at the driveline exit site on the individual.

In another embodiment, the present disclosure is directed to a method of preventing or treating a driveline infection in an individual. The method comprises: (i) introducing a blue light phototherapy system at a driveline exit site on an individual wherein the system comprises a biocompatible polymeric structure including a light emission source capable of providing light in the range of from 405 nanometers to 475 nanometers; and (ii) energizing the light emission source to provide light in the range of from 405 nanometers to 475 nanometers at the driveline exit site on the individual.

### BRIEF DESCRIPTION OF THE DRAWINGS

FIG. 1 illustrates a conventional left ventricular assist device system.

FIG. 2 illustrates a multi-structure blue light phototherapy system of the present disclosure including a standoff base structure and cap structure.

FIG. 3 is a partial cut away view of the cap structure illustrated in FIG. 2.

FIG. 4 illustrates a single structure blue light phototherapy system of the present disclosure.

FIG. 5 illustrates a bottom up view of the single structure blue light phototherapy system of FIG. 4.

FIG. 6 is a flow diagram of a method of preventing or treating a driveline infection in an individual.

### DETAILED DESCRIPTION OF THE DISCLOSURE

A left ventricular assist device (LVAD) is a pump that is surgically implanted just below the heart. One end of the device is attached to the left ventricle, which is the chamber of the heart that pumps blood out of the heart and into the body. The other end of the device is attached to the aorta, which is the body's main artery. With the LVAD, blood flows from the heart into the pump. When sensors located in the pump indicate that the LVAD is full, the blood in the device is moved into the aorta. In most embodiments, a tube passes from the device through the skin. This tube is commonly referred to as a driveline, and it connects the pump to an external controller and power source used to drive and control the pump.

Ventricular assist device driveline infections may occur in some patients at any time during the use of the ventricular assist device. In some cases, such infections may complicate treatment of the patient to a desired outcome and require specialized treatment of the infected site. To date, such treatment has included antibiotics and other medicines. The present disclosure provides suitable systems and methods for both preventing and treating undesirable ventricular driveline infections, including a wide variety of bacterial infections.

In some embodiments, the present disclosure provides a blue light phototherapy system or a blue light infection control/treatment system and methods of using the system to prevent and treat driveline bacterial infections, and particularly ventricular assist device driveline bacterial infections (Right Ventricular Assist Devices (RVAD) and Left Ventricular Assist Devices (LVAD)). The disclosed blue light phototherapy systems utilize photodynamic therapy and provide for constant, repeated and/or periodic (intermittent) antimicrobial treatment of soft tissue surrounding the driveline exit site on an individual. In many embodiments, the system includes one or more arrays of light emitting diodes (LED) of predominantly blue wavelengths (405 nanometers to 475 nanometers) in combination with a single or multi-piece biocompatible structure that captures the arrays and provides for a fixed distance from the arrays to the exit site tissue surface. The biocompatible structures and LED arrays are designed and configured to accommodate the placement and removal around the individual's driveline exit site, and may be used for acute and chronic wound management by photodynamically inactivating a large spectrum of bacteria. In many embodiments, the systems may additionally include one or more driveline cable protection members to shield and protect the ventricular assist device driveline from the LED arrays (or other suitable light emitting sources) to prevent any degradation of the driveline over time. The systems and methods of the present disclosure may be used for both preventative treatment post-operative and for the treatment/care of existing driveline infections. The systems and methods of the present disclosure may be used alone or in combination with other therapies such as antibiotics or other medicines. Although the present disclosure is primarily discussed in terms of driveline infections with LVADs, it will be recognized by one skilled in the art based on the disclosure herein that the systems and methods described herein may be equally applicable to RVADs, as well as other medical devices that utilize a driveline or other device that passes through an individual's skin.

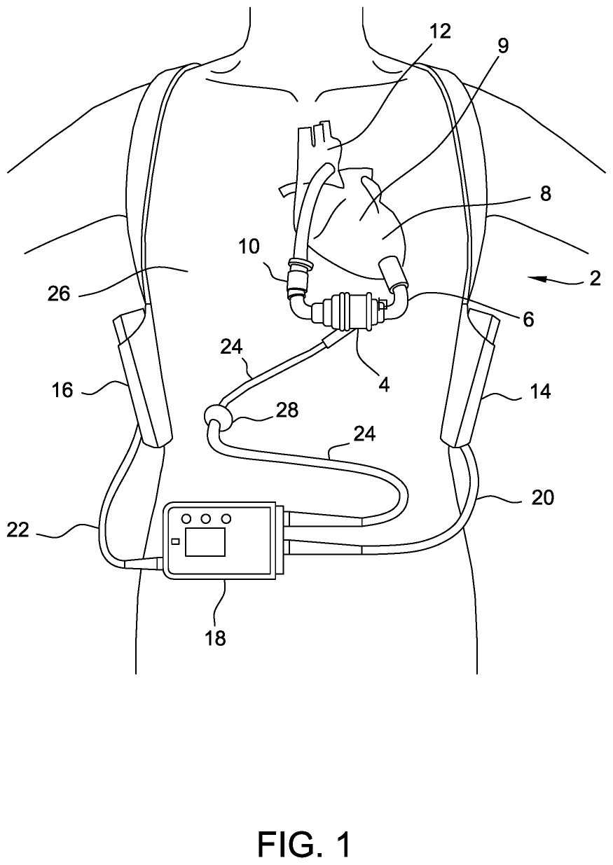

Referring now to FIG. 1, there is shown a conventional LVAD system 2. LVAD system 2 includes LVAD pump 4, outflow conduit 6 which receives blood from left ventricle 8 of heart 9, and inflow conduit 10 which provides blood to aorta 12 of heart 9. LVAD system 2 additionally includes battery packs 14 and 16 electrically connected to control unit 18 via electrical connections 20 and 22. LVAD pump 4 is electrically connected to control unit 18 via driveline 24. Driveline 24 enters body 26 at percutaneous access site 28. Percutaneous access site 28 is the location at and around which infection many occur over time.

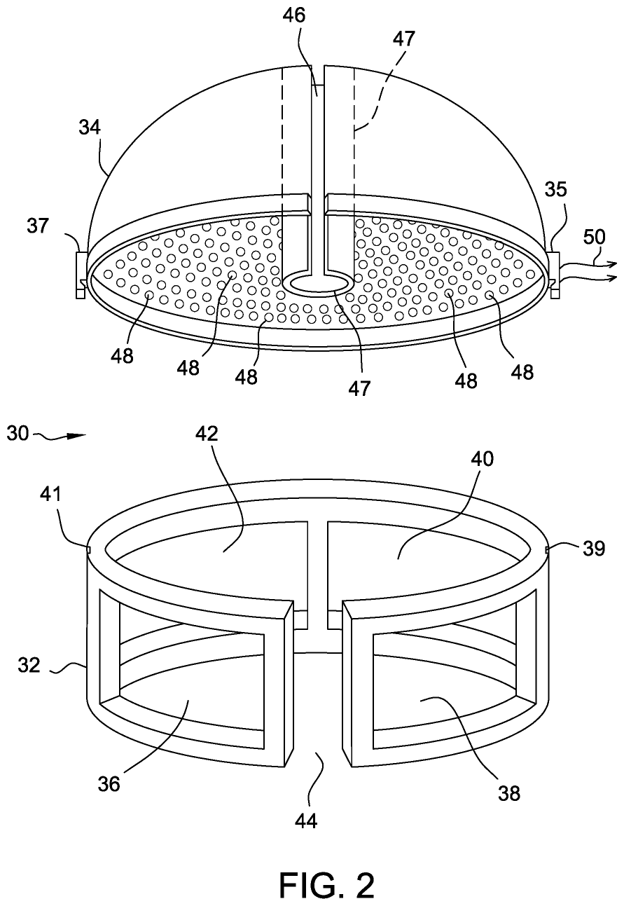

In accordance with the present disclosure, a blue light phototherapy system and kit is provided to prevent and/or treat various types of driveline or driveline-related infections that may occur in and around the area where the driveline for an LVAD system extends from the patient. Referring now to FIG. 2, in one embodiment, a blue light phototherapy system 30 includes a two piece system including a standoff base structure 32 and a cap structure 34. Standoff base structure 32 may be sized and configured for direct attachment to the skin of a patient, although embodiments where the standoff base structure is not attached to the skin are within the present disclosure. Cap structure 34 is sized and configured for attachment to standoff base structure 32 for use. Cap structure 34 may be sized and configured to attach to standoff base structure 32 in any suitable manner including, for example, a snap fit arrangement or other locking-type connection arrangement. FIG. 2 illustrates a snap fit arrangement wherein snaps 35 and 37 located on cap structure 34 snap into grooves 39 and 41 respectively located on standoff base structure 32 to secure cap structure 34 and standoff base structure 32 together for use.

Standoff base structure 32 and cap structure 34 are sized and configured for insertion and removal around a driveline cable at the driveline exit site of an individual. In FIG. 2, standoff base structure 32 is illustrated including multiple openings or windows 36, 38, 40, and 42. These multiple openings or windows allow for improved dissipation of any heat generated by a light emission source (see below) present in the blue light phototherapy system or kit. Other suitable embodiments of the present disclosure may include more or fewer or zero openings or windows.

With continued reference to FIG. 2, standoff base structure 32 includes a driveline cable slot 44 and cap structure 34 includes a driveline cable slot 46. Driveline cable slots 44 and 46 allow for standoff base structure 32 and cap structure 34, when connected together to form a single piece, to have a driveline cable (not shown in FIG. 2, but see FIG. 1) to enter therethrough and be properly positioned and secured on a patient. Additionally, cap structure 34 may optionally include driveline cable protection member 47 that is sized and configured to accommodate a driveline cable (not shown in FIG. 2, but see FIG. 1) to provide protection to the driveline cable from any degradation due to a light emission source (see below). In many embodiments, driveline cable protection member 47 may extend from cap structure 34 and into standoff base structure 32 to a driveline exit site (not shown in FIG. 2 but see FIG. 1) of a patient. Cap structure 34 additionally includes a light emission source 48 capable of providing constant or periodic light in the range of from 405 nanometers to 475 nanometers. It is this light emission source 48 that provides the blue light for the blue light phototherapy system 30. Although any suitable light emission source 48 may be utilized in accordance with the present disclosure, one desirable light emission source 48 includes one or more light emitting diode (LED) arrays. Light emission source 48 is electrically connected to a power source (not shown in FIG. 2) via electrical cable 50.

Standoff base structure 32 and cap structure 34 may be manufactured from any material suitable for use when in contact with the human body that provides a desired structural integrity. In some desirable embodiments, standoff base structure 32 and cap structure 34 may be manufactured from the same or different suitable polymeric or thermoplastic materials, including suitable biocompatible polymeric materials. In many embodiments, the materials used to construct standoff base structure 32 and cap structure 34 may be suitable for re-sterilization and reuse so as to increase the usability of the structures over time. Standoff base structure 32 and cap structure 34 may be of any suitable size, and in some embodiments may have a diameter of from about 3 inches (about 7.6 centimeters) to about 5 inches (about 12.7 centimeters), although other sized configurations are within the scope of the present disclosure.

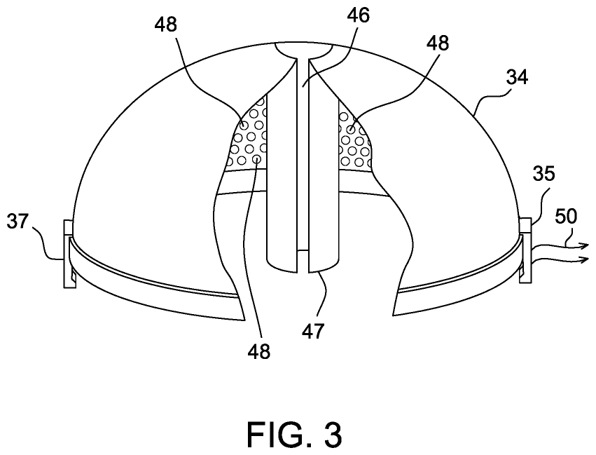

Referring now to FIG. 3, there is shown a partial cut away view of cap structure 34 as shown in FIG. 2. Cap structure 34 as illustrated in FIG. 3 includes snaps 35 and 37 located on cap structure 34 that are sized and configured to snap into grooves located on a standoff base structure (not shown in FIG. 3 but see FIG. 2) to provide a secure and tight fit between cap structure 34 and a standoff base structure. Cap structure 34 also includes a driveline cable slot 46 to allow for a driveline cable (not shown in FIG. 3 but see FIG. 1) to be easily inserted therein and driveline protection member 47 to protect a driveline from exposure to blue light. Cap structure 34 additionally includes electrical cable 50 which can be connected to a power source (not shown in FIG. 3) to power light emission source 48 located on cap structure 34.

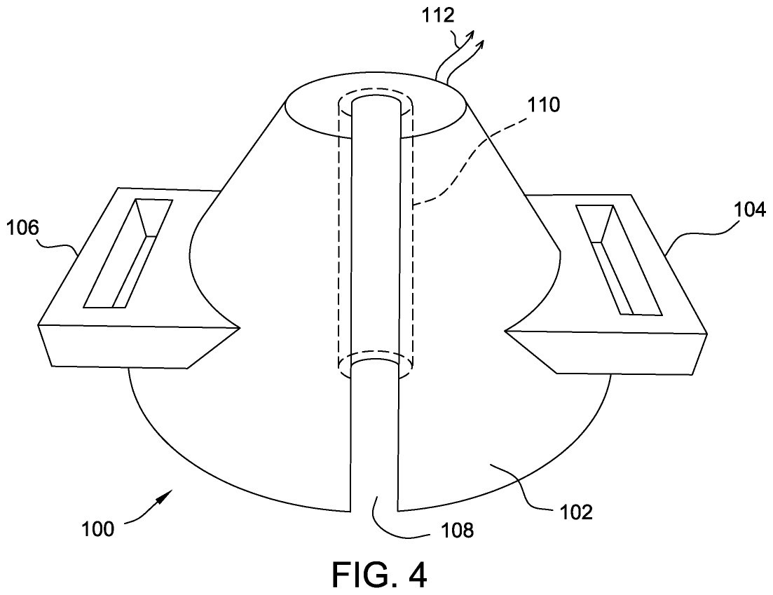

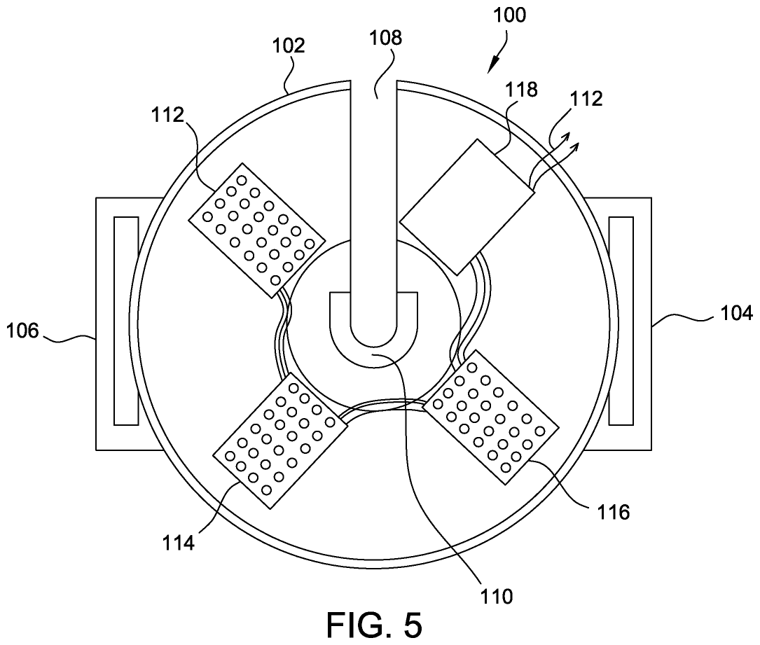

In an alternative embodiment of the present disclosure, a blue light phototherapy system suitable for treating or preventing driveline infections may include a single piece unit in place of the dual piece standoff base structure and cap structure as described above. In this alternative embodiment, the blue light phototherapy system includes a single molded or otherwise formed polymeric, thermoplastic or the like material that may include some or all of the features described above in relation to the two piece system that snap fit or otherwise connects together prior to use. This single piece structure design may be desirable for some applications. Referring now to FIG. 4, there is shown a blue light phototherapy system 100 including cap structure 102 having handles 104 and 106 to allow a user to easily move and place blue light phototherapy system 100 at a desired location. Blue light phototherapy system 100 additionally includes driveline cable slot 108 and driveline cable protection member 110. Electrical cable 112 can be connected to a power source (not shown in FIG. 4) to power a light emission source (not shown in FIG. 4 but see FIG. 5) located on cap structure 102. Additionally, blue light phototherapy system 100 may optionally include one or more openings or windows (not shown in FIG. 4 but see FIG. 2) in cap structure 102 to improve dissipation of any heat generated by a light emission source present in the blue light phototherapy system. Although many of the embodiments described and illustrated herein include an electrical cable suitable for connection to a power source to power the light emission source, it will be recognized by one skilled in the art based on the disclosure herein that the electrical cable may be completely internal and may be connected to a battery power source and/or another power source.

Referring now to FIG. 5, there is shown a bottom up view of the blue light phototherapy system 100 of FIG. 4 to illustrate further internal details. As shown in FIG. 5, blue light phototherapy system 100 includes cap structure 102 having handles 104 and 106, driveline cable slot 108 and driveline cable protection member 110. Cap structure 102 additionally includes light emission sources 112, 114, and 116 electrically connected to power module 118. Power module 118 is electrically connected to electrical cable 112 which may be connected to a power source (not shown in FIG. 5) to power light emission sources 112, 114, and 116 located on cap structure 102. Other electrical and power arrangements to power light emission sources 112, 114, and 116 are also within the scope of the present disclosure.

The present disclosure is further directed to methods and processes for preventing, treating, combating or otherwise addressing a driveline or driveline-related infection in an individual and, in particular, a driveline infection in relation to the use of a ventricular assist device such as a left ventricular assist device. In one embodiment, the method of preventing or treating a driveline infection includes (i) introducing a blue light phototherapy system at a driveline exit site on an individual wherein the blue light phototherapy system includes a biocompatible polymeric structure including a light emission source capable of providing light in the range of from 405 nanometers to 475 nanometers; and (ii) energizing the light emission source to provide light in the range of from 405 nanometers to 475 nanometers at the driveline exit site on the individual. In many embodiments, the blue light phototherapy system will be energized at the driveline exit site for from about 1 minute to about 3 hours to prevent or treat a driveline infection. Such application of the blue light at the driveline exit site may be performed one or more times daily and may be performed from 1 day to several weeks depending upon the amount of therapy required by an individual. The blue light phototherapy systems described herein for use at a driveline exit site on an individual may be used as a sole method for preventing and/or treating a driveline infection, or may be used in combination with alternative therapies for preventing and/or treating a driveline infection including, for example, antibiotics, other medicines, and the like. Additionally, the blue light phototherapy systems of the present disclosure may provide therapeutic light to the driveline exit site on an individual in a constant or intermittent manner, and may be programmed on a desirable time and frequency duration basis.

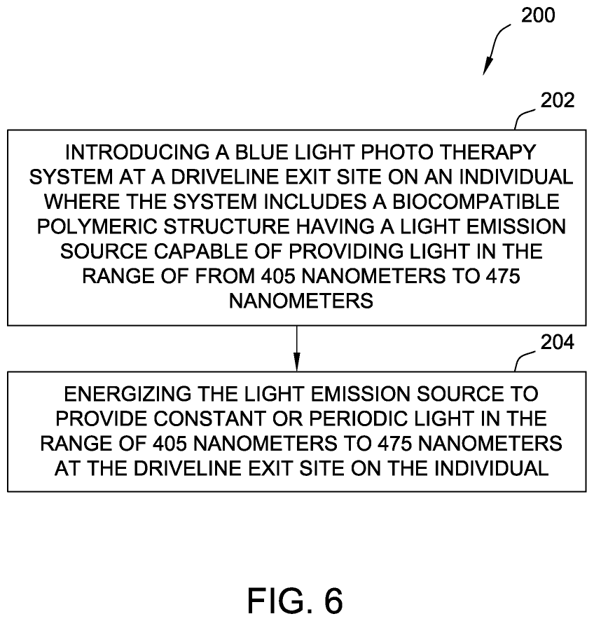

Referring now to FIG. 6, there is shown a flow diagram of a method 200 for preventing or treating a driveline infection in an individual. Notably, the steps in method 200 may be performed in any suitable order, and are not limited to being performed in the order shown in FIG. 6. Method 200 includes introducing 202 a blue light phototherapy system at a driveline exit site on an individual where the system includes a biocompatible polymeric structure having a light emission source capable of providing light in the range of from 405 nanometers to 475 nanometers and energizing 204 the light emission source to provide constant or periodic light in the range of 405 nanometers to 475 nanometers at the driveline exit site on the individual.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A blue light phototherapy system for treating or preventing an infection at a driveline exit site on an individual, the system comprising:
a standoff base structure sized and configured for insertion and removal around a driveline cable at the driveline exit site of the individual; and
a cap structure sized and configured for attachment to the standoff base structure, the cap structure including a light emission source capable of providing light in the range of from 405 nanometers to 475 nanometers.

2. The system of claim 1, wherein the standoff base structure and the cap structure both include a driveline cable slot.

3. The system of claim 1, wherein the cap structure includes a driveline cable protection member sized and configured to accommodate the driveline cable to protect the driveline cable from the light emission source.

4. The system of claim 3, wherein the driveline cable protection member extends from the cap structure and into the standoff base structure to the driveline exit site.

5. The system of claim 1, wherein the standoff base structure has a diameter of from about 3 to about 5 inches.

6. The system of claim 1, wherein the standoff base structure and the cap structure are both formed of a biocompatible polymeric material.

7. The system of claim 1, wherein the cap structure is attached to the standoff base structure via snap fit arrangement.

8. The system of claim 1, wherein the standoff base structure is suitable for re-use and re-sterilization.

9. The system of claim 1, wherein the standoff base structure includes one or more openings therein to allow for the dissipation of heat generated by the light emission source.

10. The system of claim 1, wherein the light emission source includes one or more light emitting diode arrays.

11. The system of claim 1, wherein the standoff base structure is sized and configured for attachment to skin.

12. A blue light phototherapy system for treating or preventing an infection at a driveline exit site on an individual, the system comprising:
a biocompatible polymeric structure sized and configured for insertion and removal around a driveline cable at the driveline exit site of the individual;
wherein the biocompatible polymeric structure includes a light emission source capable of providing light in the range of from 405 nanometers to 475 nanometers at the driveline exit site on the individual.

13. The system of claim 12, wherein the biocompatible polymeric structure includes a driveline cable slot.

14. The system of claim 13, wherein the biocompatible polymeric structure includes a driveline cable protection member sized and configured to accommodate the driveline cable to protect the driveline cable from the light emission source.

15. The system of claim 12, wherein the biocompatible polymeric structure has a diameter of from about 3 to about 5 inches.

16. The system of claim 12, wherein the biocompatible polymeric structure is suitable for re-use and re-sterilization.

17. The system of claim 12, wherein the biocompatible polymeric structure includes one or more openings therein to allow for the dissipation of heat generated by the light emission source.

18. The system of claim 12, wherein the light emission source includes one or more light emitting diode arrays.

19. The system of claim 12, wherein the biocompatible polymeric structure is sized and configured for attachment to skin.

20. A method of preventing or treating a driveline infection in an individual, the method comprising:
introducing a blue light phototherapy system at a driveline exit site on an individual, the system comprising a biocompatible polymeric structure including a light emission source capable of providing light in the range of from 405 nanometers to 475 nanometers; and
energizing the light emission source to provide light in the range of from 405 nanometers to 475 nanometers at the driveline exit site on the individual.
